(19) United States
(12) Patent Application Publication
(10) Pub. No.: US 2006/0004321 A1
(43) Pub. Date: Jan. 5, 2006

(54) Painless drug delivery electrode device

(75) Inventors: Tak-Wah Wong, Kaohsiung City (TW); Sek-Wen Hui, Williamsville, NY (US); Ching-Hung Chen, Yung-Kang City (TW); Chien-Chun Huang, Tainan City (TW); Cheng-De Lin, Chang-Hua Hsien (TW)

Correspondence Address: Paul D. Greeley, Esq.;Ohlandt, Greeley, Ruggiero & Perle, L.L.P., 10th Floor, One Landmark Square, Stamford, CT, 06901-2682, US

(73) Assignee: National Cheng Kung University

(21) Appl. No.: 11/169,874

(22) Filed: Jun. 29, 2005

(30) Foreign Application Priority Data
Jun. 30, 2004 (TW) 093119711

(51) Int. Cl. A61N 1/30

(52) U.S. Cl. 604/20

(57) ABSTRACT

A painless drug delivery electrode device includes a substrate having top and bottom sides, and an electrode unit provided on the top side of the substrate and adapted to convey pulse signals to a skin. The electrode unit includes a plurality of positive and negative electrode pads which are adapted to contact the skin and which are arranged in rows. Each of the positive and negative electrode pads has a skin contact surface area smaller than 1 sq. mm. Each of the positive electrode pads is spaced apart from an adjacent one of the negative electrodes by a distance ranging from 0.2-1 mm.

### CROSS-REFERENCE TO RELATED APPLICATION

This application claims priority of Taiwanese Application No. 93119711, filed on Jun. 30, 2004.

### BACKGROUND OF THE INVENTION

1. Field of the Invention

The invention relates to a drug delivery electrode device, more particularly to a painless drug delivery electrode device that is used to form electropores in a patient's skin in a painless manner so as to permit subsequent transdermal drug administration through the electropores.

2. Description of the Related Art

Administration of therapeutic drugs to patients generally includes oral administration, injection, and transdermal/transmucosal administration. However, oral drugs may cause stomach irritation, whereas injection is painful to the patient. Therefore, the medical field has been endeavoring to develop methods of administering drugs to patients through skin. However, since skin is the most important barrier against bacteria and viruses from invading the human body, therapeutic drugs in general cannot be easily absorbed. In particular, stratum corneum of the skin is a main barrier against absorption of drugs. Therefore, some scholars have developed a method called iontophoresis in which the drug is applied onto the surface of the skin, and an electrode device is used to apply an electric current to the skin such that the drug enters into the human body through electrophoresis and/or electro-osmosis. However, this method is disadvantageous in that the intact skin barrier will obstruct the transport of large quantity or large size drug molecules into the human body so that the therapeutic effect is usually not satisfactory. Besides, the drug may be changed chemically due to electrolysis.

Another method is electroporation, in which electropores are formed in the epidermal, dermal and subcutaneous cells using an invasive electrode, and the drug is delivered directly through the electropores into the cells and intercellular spaces. However, the high voltage electric pulses are transmitted directly to nerve cells of the skin and the muscle, which can induce pain and muscle contraction. Thus, this method is not widely adopted.

### SUMMARY OF THE INVENTION

Therefore, an object of the present invention is to provide an electrode device that can be used to form electropores in the stratum corneum of a patient's skin in a painless manner so as to permit subsequent transdermal drug administration through the electropores.

Accordingly, a painless drug delivery electrode device includes a substrate having top and bottom sides, and an electrode unit provided on the top side of the substrate and adapted to generate pulse signals to the skin. The electrode unit includes a plurality of positive and negative electrode pads which are adapted to contact the skin and which are arranged in rows. Each of the positive and negative electrode pads has a skin contact surface area smaller than 1 sq. mm. Each of the positive electrode pads is spaced apart from an adjacent one of the negative electrodes by a distance ranging from 0.2-1 mm.

### BRIEF DESCRIPTION OF THE DRAWINGS

Other features and advantages of the present invention will become apparent in the following detailed description of the preferred embodiment with reference to the accompanying drawings, of which:

FIG. 1 is a perspective view of the preferred embodiment of an electrode device according to the invention;

FIG. 2 is a top view of the preferred embodiment;

FIG. 3 is a fragmentary sectional view taken along line III-III of FIG. 2, illustrating the flow of electric pulse signals through the stratum corneum when a plurality of positive and negative electrode pads are disposed to contact a patient's skin;

FIG. 4 is a fragmentary sectional view taken along line IV-IV of FIG. 2, showing the connection among a positive conducting member, a positive connecting plate, and the positive electrode pads;

FIG. 5 is a picture showing residual drug on the skin of a mouse when the preferred embodiment is employed in an in vivo experiment;

FIG. 6 is a picture showing the permeation of the drug through an electropore in the mousers skin into the adipose tissues; and

FIGS. 7-11 are schematic views showing the preferred embodiment of a method for fabricating an electrode device according to this invention.

### DETAILED DESCRIPTION OF THE PREFERRED EMBODIMENT

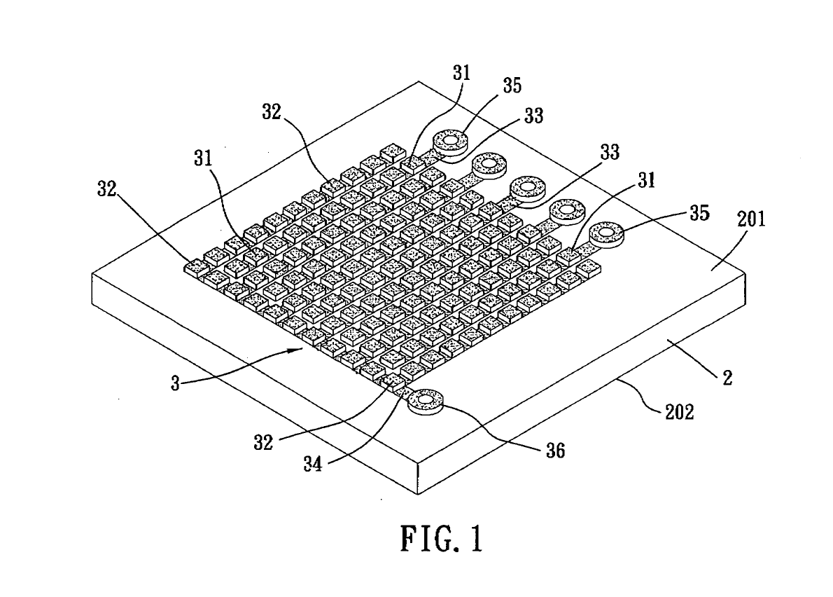

Referring to FIGS. 1, 2 and 3, the preferred embodiment of a drug delivery electrode device according to the present invention is shown to be adapted to transmit a single or a continuous train of electric pulse signal to the surface of skin 1 of a patient such that a plurality of electropores 12 are formed in the stratum corneum 11 of the skin 1 for subsequent application of a drug to the surface of the skin 1 such that the drug permeates into the body of the patient through the electropores 12. The preferred embodiment includes a square-shaped substrate 2 formed from an electrical insulating material, and an electrode unit 3 provided on the substrate 2.

The electrode unit 3 conducts the electric pulse signal to the surface of the skin 1. The electrode unit 3 includes a plurality of positive electrode pads 31 and a plurality of negative electrode pads 32 arranged in rows and in an alternating manner on a top side 201 of the substrate 2, a plurality of positive connecting plates 33 formed on the top side 201 of the substrate 2 and electrically connected to all the positive electrode pads 31 in each row, one negative connecting plate 34 formed on the top side 201 of the substrate 2 and connected electrically to all rows of the negative electrode pads 32, a plurality of positive conducting members 35 each extending through the top side 201 and a bottom side 202 of the substrate 2 and connected electrically and respectively to the positive electrode pads 31 in each row through the corresponding positive connecting plate 33, and one negative conducting member 36 which extends through the top side 201 and the bottom side 202 of the substrate 2, which is connected electrically and respectively to all rows of the negative electrode pads 32 through the negative connecting plate 34, and which projects outwardly of the bottom side 202 of the substrate 2.

Preferably, each of the positive and negative electrode pads 31, 32 has a skin contact surface area smaller than 1 sq. mm, and each positive electrode pad 31 is spaced apart from an adjacent negative electrode pad 32 by a distance ranging from 0.2-1 mm. Based on actual experiments, if the spacing between the positive and negative electrode pads 31, 32 of the electrode unit 3 is less than 0.3 mm, short-circuit between the positive and negative electrode pads 31, 32 may result. However, if the spacing is greater than 0.3 mm, the pain caused to the patient's skin will increase. Furthermore, the size of the positive and negative electrode pads 31, 32 is also a factor that can influence the amount of pain during application of the electric pulses. In addition, the positive and negative electrode pads 31, 32 are configured to be square in shape in this embodiment for purposes of simplifying fabrication, but should not be limited thereto in practice. Moreover, the thickness of the positive and negative electrode pads 31, 32 is configured to be 0.2 mm so that the positive and negative electrode pads 31, 32 will not create a prickly sensation to the patient's skin when in contact therewith. Thus, in this embodiment, the positive and negative electrode pads 31, 32 are configured to have a square shape with each side having a dimension of about 0.5 mm, and a height/thickness of about 0.2 mm. The spacing between each positive electrode pad 31 and an adjacent negative or positive electrode pad 31, 32 is maintained at 0.3 mm.

In this embodiment, the positive electrode pads 31 are arranged in five rows. There are ten positive electrode pads 31 in each row. Five positive conducting members 35 are disposed respectively at front ends of the five rows of positive electrode pads 31. The positive electrode pads 31 in each row are connected to one of the positive conducting members 35 at the front end thereof by a respective one of the positive connecting plates 33 so as to be interconnected electrically.

The negative electrode pads 32 are arranged in six rows. There are eleven negative electrode pads 32 in each row. The six rows of negative electrode pads 32 are arranged alternately with the five rows of positive electrode pads 31. The six rows of negative electrode pads 32 are interconnected electrically through the negative connecting plate 34, and are connected to the negative conducting member 36, which is disposed at a rear edge of the substrate 2.

In the present invention as exemplified hereinabove, five positive connecting plates 33 are used to connect the five rows of positive electrode pads 31 to the respective positive conducting members 35. However, it should be noted that all the positive electrode pads 31 can be connected to only one positive conducting member 35 by a single positive connecting plate 33. Therefore, the number and arrangement of the positive and negative electrode pads 31, 32 can be varied depending on drug administration requirements, and should not be limited to the foregoing. In addition, the manner of connection of the positive and negative connecting plates 33, 34 can be adjusted to enable electrical connection of some or all of the positive and negative electrode pads 31, 32.

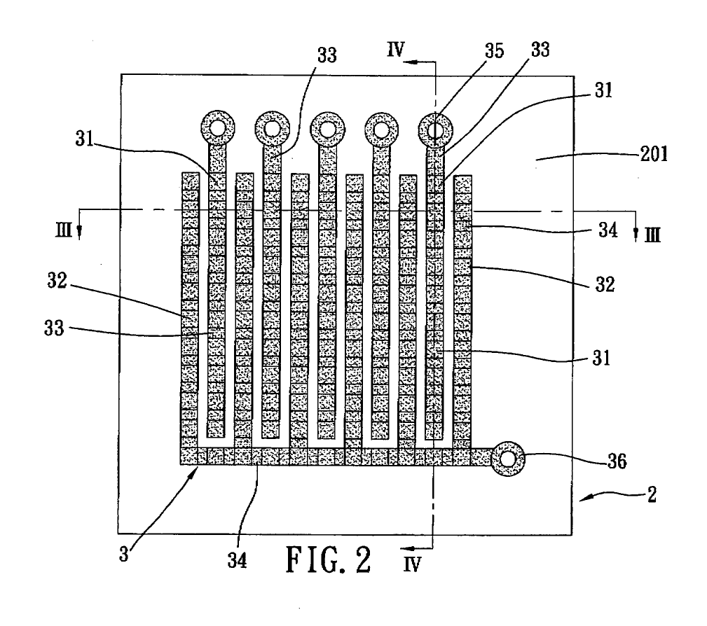

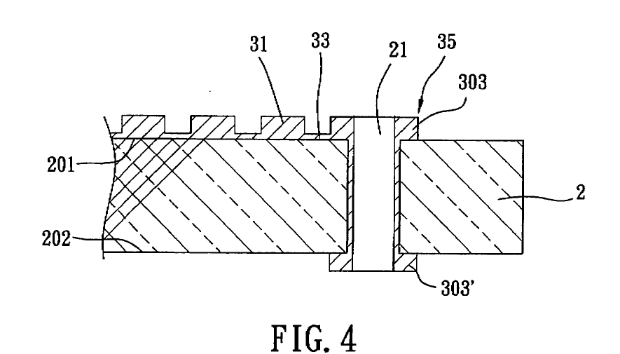

Referring to FIG. 4 in combination with FIG. 2, the positive and negative conducting members 35, 36 are hollow tubular structures. The electric pulse signal is conducted from the bottom side 202 of the substrate 2 to the top side 201 of the substrate 2 for transmission to the positive and negative electrode pads 31, 32 through the positive and negative conducting plates 33, 34. FIG. 4 illustrates the structures and connective relationship of the positive conducting member 35, the positive connecting plate 33, and the positive electrode pads 31. The structures and connective relationship of the negative conducting member 36, the negative connecting plate 34, and the negative electrode pads 32 are not illustrated therein as they are substantially similar to those of the positive conducting member 35, the positive connecting plate 33, and the positive electrode pads 31.

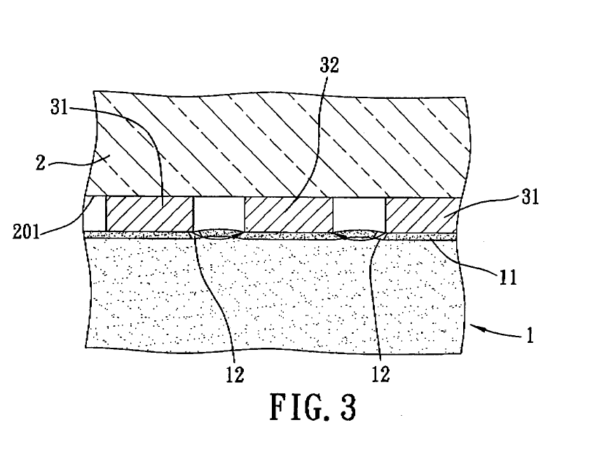

Referring once again to FIG. 3, in use, the top side 201 of the substrate 2 is pressed against the surface of the skin 1 such that the positive and negative electrode pads 31, 32 contact the surface of the skin 1. Then, the electric pulse signals are conducted to the positive conducting members 35 which project from the bottom side 202 of the substrate 2, whereas the negative conducting member 36 is grounded. When two or more electric pulse signals are transmitted in succession to the surface of the skin 1, the first electric pulse signal will create an electropore 12 in the stratum corneum 11 of the skin 1, whereas the second electric pulse signal will maintain the state of the electropore 12.

Since the area of each positive electrode pads 31 is less than 1 sq. mm., the area of contact between each positive electrode pad 31 and the skin 1 is very small. In addition, since the spacing between the positive and negative electrode pads is only 0.3 mm, and since the duration of the pulse signal is only 0.2 milliseconds, the electric currents from the positive electrode pads 31 will only penetrate into the stratum corneum 11 of the skin 1, which is the outermost layer of the skin 1 and which has the largest resistance, and will leave the skin 1 through the shortest route (i.e., through the respective negative electrode pads 3 closest thereto) to return to the negative electrode pads 32 for grounding, thereby forming a closed circuit loop. Thus, the electric currents of the electric pulse signals will only enter into relatively shallow areas of the skin 1 and will not reach the threshold of the sensory nerve response so that pain will not be felt in the skin 1. Besides, the electric currents of the electric pulse signals will not flow through the muscular tissues and, therefore, will not cause muscular contraction. The electric pulse signals may be square wave pulses, exponential decay pulses, or AC pulses. The voltage is at least more than 50V. Each pulse is maintained for a duration of less than 1 second. The interval between pulses is less than 5 seconds.

The effects of this invention will be illustrated by way of an exemplary in vivo experiment conducted on mice, as follows:

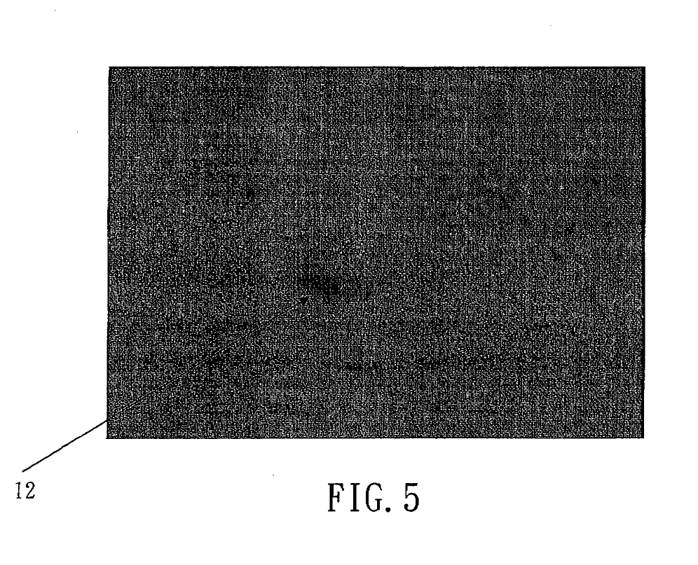

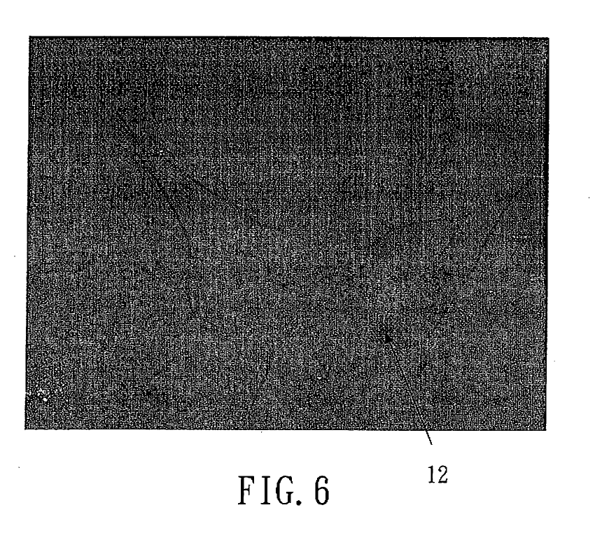

First, the positive and negative electrode pads 31, 32 were placed on the epidermis of the skin 1 of the mice, and electric pulse signals (pulse amplitude: 150V; pulse width: 0.2 ms, pulse interval: 0.1 sec.; a total of 180 pulses) were applied for a duration of 20 seconds to form electropores in the skin 1 of the mice. Thereafter, toluidine Blue O was applied to the epidermis and was allowed to stay for 30 minutes before rinsing. The picture illustrated in FIG. 5 shows that residual blue drug was found only at the electropore 12. The picture in FIG. 6 shows that the blue drug passes through the skin of the mouse into the adipose layer. Although the drug applied covered an area of about 1 cm², the drug permeated into the skin of the mouse only through the electropore 12. This demonstrates that toluidine Blue O permeated into the body of the mouse through the electropore 12.

Subsequently, a plurality of mice were divided into two groups. One was the experimental group. The other was the control group. Electropores were formed in the skins of the mice in the experimental group in the same manner as described hereinabove. Methotrexate, a chemotherapy drug, was applied to the surface of the skin 1 of the mice in the experimental group and was allowed to stay for 30 minutes. Thereafter, the surface of the skin 1 was washed with water for 1 minute to remove residual drug on the skin 1. The mice were allowed to rest for 30 minutes before blood was extracted therefrom for blood tests. For the control group, methotrexate was applied directly to the skin 1 of the mice without electroporation. After 30 minutes, the surface of the skin 1 was washed with water for 1 minute. The mice were allowed to rest for 30 minutes before blood was extracted therefrom for blood tests.

After comparing the blood of the mice in the experimental and control groups, it was found that the concentration of drug in the blood of the mice in the experimental group was higher than that in the blood of the mice in the control group by 3 folds. This demonstrated that this invention could enhance penetration of the chemotherapy drug into the bodies of the mice to thereby increase transdermal absorption of the drug.

In addition, an experiment conducted on four healthy human subjects showed that there was no incidence of muscle contraction, and the tested subjects did not feel any pain at all. Thus, it has been shown that this invention improves transdermal absorption of drugs, and will not cause any pain or discomfort.

The present invention can be adapted for administrating various therapeutic agents, such as anesthetics, antibiotics, hormones, chemotherapy agents, nucleic acid sequences, peptides, protein, various vaccine or serum combinations, etc. The present invention can also be adapted for use in plastic surgery to deliver skin care agents, botulinum toxin, or the like into the human body.

The method for fabricating the drug delivery electrode device according to this invention will be described in the succeeding paragraphs.

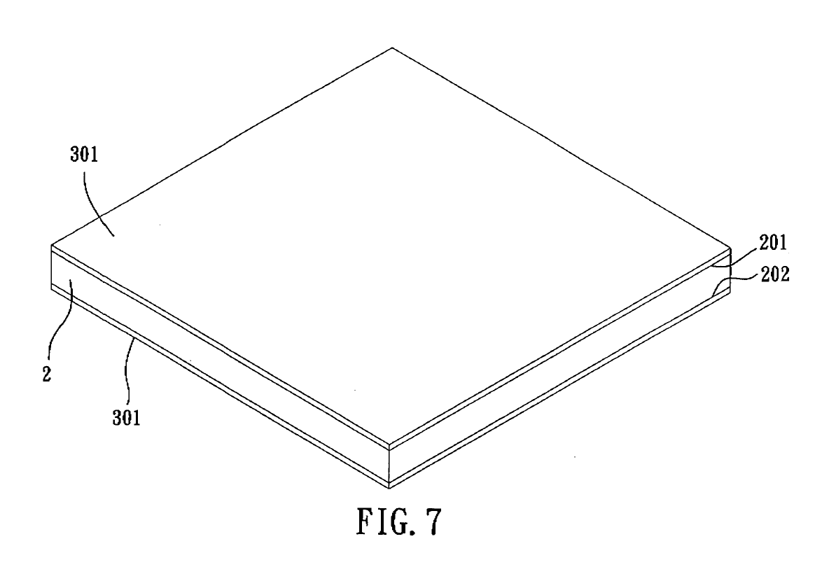

Referring to FIG. 7, a thick metal plate layer 301 having a thickness of 0.2 mm is plated on each of the top side 201 and the bottom side 202 of the insulating substrate 2. In this embodiment, the thick metal plate layer 301 is formed from copper.

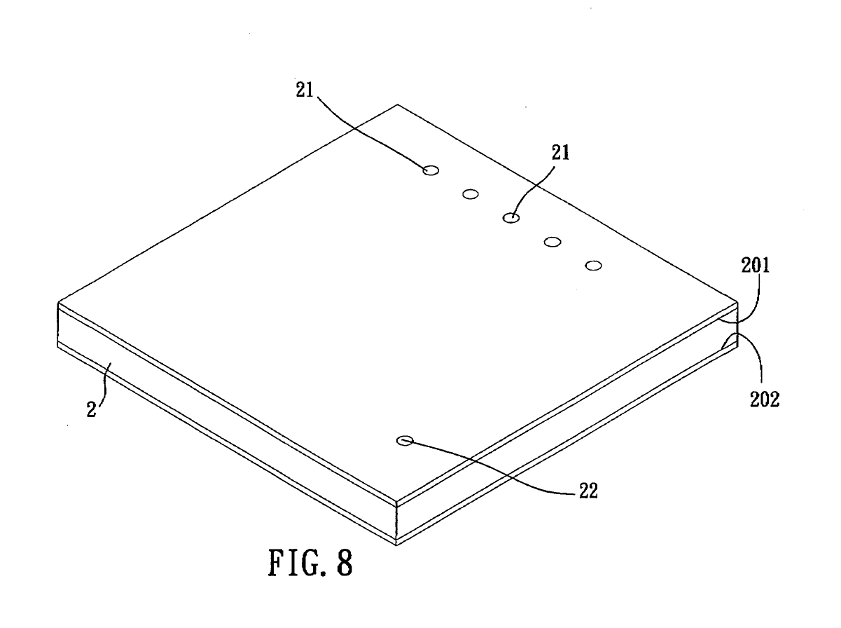

Referring to FIG. 8, a front part of the substrate 2 is drilled to form five transversely spaced-apart through holes 21. A rear part of the substrate 2 is drilled to form a through hole 22.

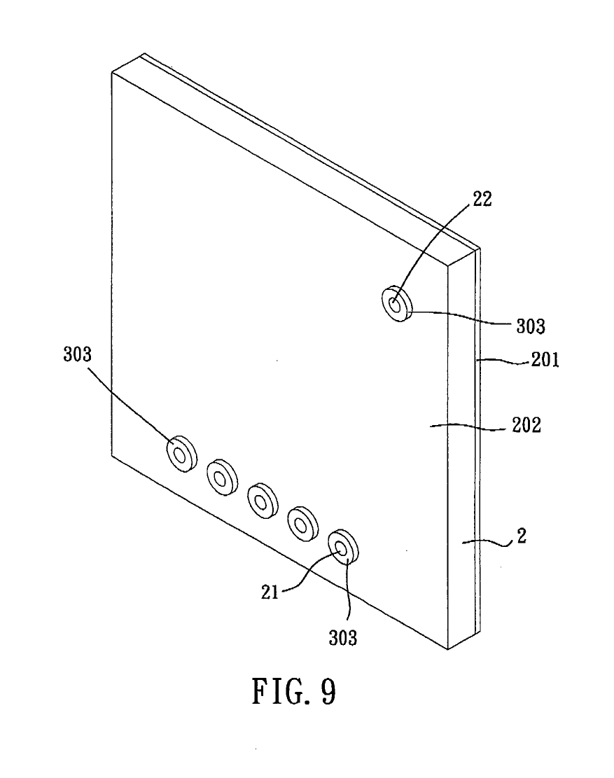

Referring to FIG. 9, the thick metal plate layer 301 on the bottom side 202 of the substrate 2 is removed in part by etching or engraving such that a ring-shaped metal plate 303′ is formed around each of the through holes 21, 22 on the bottom side 202.

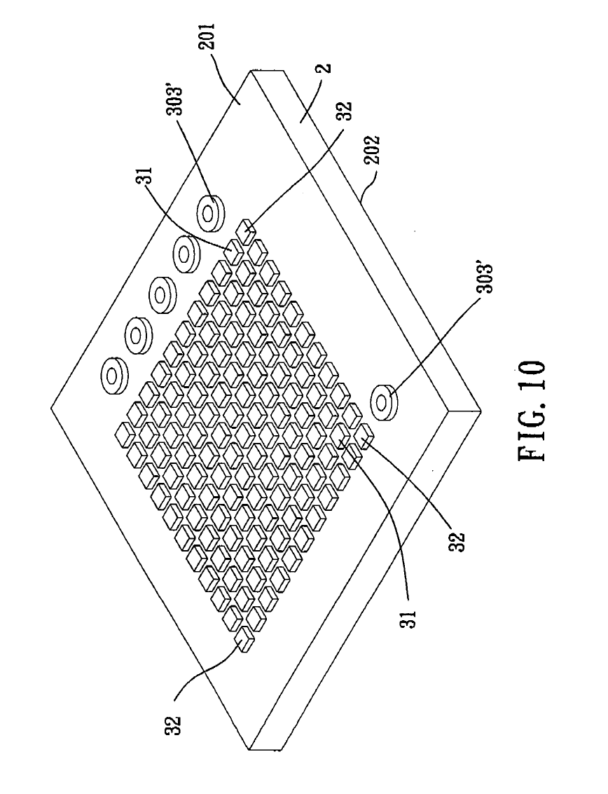

Referring to FIG. 10, the thick metal plate layer 301 on the top side 201 of the substrate 2 is engraved using an engraving machine (not shown) to form the positive electrode pads 31, the negative electrode pads 32, and ring-shaped metal plates 303 around the through holes 21, 22 on the top side 201 such that the positive and negative electrode pads 31, 32 are isolated electrically from each other and are alternately arranged.

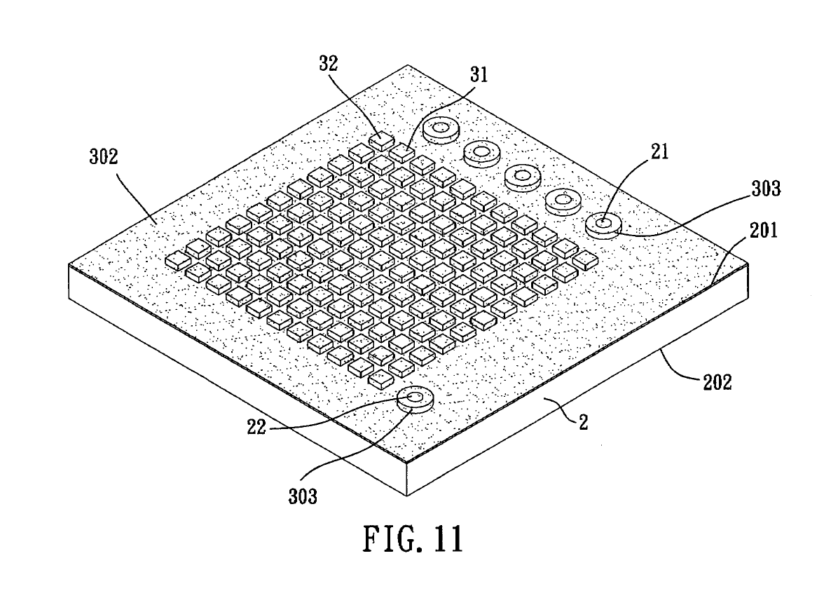

Referring to FIG. 11, a thin metal layer 302 is electroplated on the top side 201 of the substrate 2 using copper sulfate as the electroplating solution such that inner walls defining the through holes 21, 22 are also plated with the thin metal layer 302 to enable electrical connection between the metal plates 303, 303′ on the top and bottom sides 201, 202 of the substrate 2, thereby forming the positive and negative conducting members 35, 36 as shown in FIG. 1.

Thereafter, the thin metal plate layer 302 between each positive electrode pad 31 and the negative electrode pad(s) 32 adjacent thereto is removed by etching such that each row of the positive electrode pads 31 is connected electrically to the respective positive conducting member 35 and such that each row of the negative electrode pads 32 is connected electrically to the negative conducting member 36, with the positive and negative electrode pads 31, 32 isolated electrically from each other.

The process of forming the drug delivery electrode device according to this invention, as illustrated hereinabove, is merely an example of the method of this invention. The photo-etching process, which is a technically mature process, can be used to fabricate the device of this invention at reduced costs so that the electrode device can be discarded after use.

In sum, this invention utilizes the ultra-small positive and negative electrode pads 31, 32, that are spaced apart by a narrow spacing to apply electric pulse signals to the surface of the skin 1 so that the electric current passes through a path that is shallow from the skin surface, and does not flow into the cellular tissues beneath the stratum corneum 11 to cause pain to the patient or muscle contraction. Besides, permeation of the drug delivered through the electropores 12 in the stratum corneum 11 can be enhanced. Since the drug is applied to the skin 1 after electroporation, the integrity of the drug can be maintained.

While the present invention has been described in connection with what is considered the most practical and preferred embodiment, it is understood that this invention is not limited to the disclosed embodiment but is intended to cover various arrangements included within the spirit and scope of the broadest interpretation so as to encompass all such modifications and equivalent arrangements.

We claim:

1. A painless drug delivery electrode device comprising:
a substrate having top and bottom sides; and
an electrode unit provided on said top side of said substrate and adapted to convey electric pulse signals to a skin, said electrode unit including a plurality of positive and negative electrode pads which are adapted to contact the skin and which are arranged in rows, each of said positive and negative electrode pads having a skin contact surface area smaller than 1 sq. mm, each of said positive electrode pads being spaced apart from an adjacent one of said negative electrodes by a distance ranging from 0.2-1 mm.

2. The painless drug delivery electrode device as claimed in claim 1, wherein said skin contact surface area of each of said positive and negative electrode pads is a square with each side having a dimension of about 0.5 mm.

3. The painless drug delivery electrode device as claimed in claim 2, wherein each of said positive electrode pads is spaced apart from the adjacent one of said negative electrode pads by a distance of about 0.3 mm.

4. The painless drug delivery electrode device as claimed in claim 1, wherein each of said positive and negative electrode pads has a height of about 0.2 mm measured from said top side of said substrate.

5. The painless drug delivery electrode device as claimed in claim 1, wherein the rows of said positive electrode pads are arranged alternately with the rows of said negative electrode pads.

6. The painless drug delivery electrode device as claimed in claim 5, wherein said electrode unit further includes a plurality of positive connecting plates and a plurality of positive conducting members, each of said connecting plates being connected to all of said positive electrode pads in each row and one of said positive conducting members.

7. The painless drug delivery electrode device as claimed in claim 6, wherein said electrode unit further includes one negative connecting plate and one negative conducting member, said negative conducting member being connected to all rows of said negative electrode pads through said negative connecting plate.

8. The painless drug delivery electrode device as claimed in claim 7, wherein each of said negative and positive conducting members includes a conductive through hole extending through said top and bottom sides, a top ring-shaped metal plate formed on said top side and connected electrically to said through hole, and a bottom ring-shaped metal plate formed on said bottom side and connected electrically to said through hole.
